(19) United States
(12) Patent Application Publication
(10) Pub. No.: US 2023/0355985 A1
(43) Pub. Date: Nov. 9, 2023

(54) NEURAL DOSE CALCULATION

(71) Applicant: Stephen T. Pyles, Ocala, FL (US)

(72) Inventor: Stephen T. Pyles, Ocala, FL (US)

(21) Appl. No.: 18/312,852

(22) Filed: May 5, 2023

Related U.S. Application Data
(60) Provisional application No. 63/364,211, filed on May 5, 2022.

(51) Int. Cl. A61N 1/36; A61N 1/372

(52) U.S. Cl. CPC A61N 1/3615; A61N 1/37247; A61N 1/36071; A61N 1/36062

(57) ABSTRACT

A pulse generator stores a neural dose prescription for releasing electrical signals to stimulate neural tissue. The pulse generator generates the electrical signals for delivery through a lead to the neural tissue, the electrical signals being programmed to mitigate symptoms by stimulating the neural tissue. The pulse generator records delivery of the electrical signals, and calculates a total amount of energy delivered to the neural tissue.

### CROSS-REFERENCE TO RELATED APPLICATION

This application claims the benefit of and priority to U.S. Provisional Patent Application No. 63/364,211, filed May 5, 2022, the disclosure of which is herein incorporated by reference in its entirety.

### BACKGROUND

Spinal cord stimulation (SCS) provides pain relief for patients suffering from chronic intractable pain. SCS uses electrical signals to relieve chronic pain of the back, arms, and legs. The electrical signals interfere with, or block pain signals received by the brain. SCS is typically performed for patients who suffer from chronic pain when other therapies have failed.

Unlike other surgical procedures, a trial SCS procedure is performed before offering a patient a permanent SCS surgical implant. The trial SCS procedure is typically preformed in the physician's office or in an outpatient surgical facility, and includes implanting temporary SCS leads into the posterior epidural space of the patient's spine using an epidural needle.

A pulse generator is an electronic device that generates the electrical signals, which are delivered through a lead into the body of a patient. A lead is typically a catheter type device containing thin wire that extends from a proximal end allowing the pulse generator to communicate with contacts positioned on a distal end of the lead. The distal end of the lead is typically placed in the epidural space located between the spinal cord and the internal posterior vertebrae. The electrical signals emitted from the contacts interacts with nerves and ganglia near the epidural space to interfere with and/or block pain signals received by the brain.

The long-term effects of the electrical signals received by the body from pulse generators, including the amount of energy delivered to the brain, spinal cord, nerves, and ganglia of the central nervous system, is not presently known at this time.

### SUMMARY

In general terms, the present disclosure relates to neurostimulation. In one configuration, a device provides a technical effect by administering a neural dose that provides a lowest effective amount of energy to stimulate neural tissue for mitigating one or more symptoms. In a further configuration, the device records delivery of electrical signals, and calculates a total amount of energy delivered to the neural tissue. Various aspects are described in this disclosure, which include, but are not limited to, the following aspects.

One aspect relates to a pulse generator comprising at least one processing device; and at least one memory storage device storing: a neural dose prescription for releasing electrical signals to stimulate neural tissue; and instructions which, when executed by the at least one processing device, cause the at least one processing device to: generate, based on the neural dose prescription, the electrical signals for delivery through a lead, the electrical signals being programmed to mitigate symptoms by stimulating the neural tissue; record delivery of the electrical signals; and calculate a total amount of energy delivered to the neural tissue based on recorded deliveries of the electrical signals.

Another aspect relates to a remote control for operating a pulse generator, the remote control comprising: at least one processing device; and at least one memory storage device storing instructions which, when executed by the at least one processing device, cause the at least one processing device to: generate a command based on a neural dose prescription; send the command to a pulse generator, the command causing the pulse generator to generate electrical signals, the electrical signals being programmed to mitigate symptoms by stimulating neural tissue; record delivery of the electrical signals; and calculate a total amount of energy delivered to the neural tissue based on recorded deliveries of the electrical signals.

Another aspect relates to a method of performing spinal cord stimulation, the method comprising: generating electrical signals based on a neural dose prescription, the electrical signals delivering a lowest effective amount of energy to mitigate symptoms by stimulating neural tissue; recording delivery of the electrical signals; and calculating a total amount of energy delivered to the neural tissue based on recorded deliveries of the electrical signals.

Another aspect relates to a method of performing spinal cord stimulation, the method comprising: receiving a selection of a neural dose prescription; generating a command based on the selection of the neural dose prescription; sending the command to a pulse generator having a lead implanted in an epidural space, the command causing the pulse generator to generate electrical signals for release by the lead in the epidural space, the electrical signals being programmed to interfere with or block pain signals based on the neural dose prescription; recording delivery of the neural dose prescription; and calculating a total amount of energy delivered to the epidural space over a predetermined period of time based on recorded deliveries of the neural dose prescription over the predetermined period of time.

### DESCRIPTION OF THE FIGURES

The following drawing figures, which form a part of this application, are illustrative of the described technology and are not meant to limit the scope of the disclosure in any manner.

FIG. 1 illustrates an example of a system for performing a procedure to implant one or more leads for spinal cord stimulation.

FIG. 2 illustrates an example of a kit for spinal cord stimulation, the kit including a pulse generator, a remote control, and leads for attachment to the pulse generator.

FIG. 3 illustrates an example of a method of monitoring spinal cord stimulation during therapeutic treatment.

FIG. 4 illustrates another example of a method of monitoring spinal cord stimulation during therapeutic treatment.

FIG. 5 illustrates an example of a screen displayed on the remote control of FIG. 2

FIG. 6 illustrates an example of a screen displayed on the remote control of FIG. 2.

FIG. 7 schematically illustrates an example of a method of performing a titration of a neural dose prescription.

FIG. 8 schematically illustrates an example of a method of automatically adjusting a neural dose prescription based on a status of a patient.

FIG. 9 schematically illustrates an example of computing components of the pulse generator, the remote control, and the leads of FIG. 2.

FIG. 10 illustrates an example of a graphical user interface that can be used by a user to prescribe a neural dose for delivery by the pulse generator of FIG. 2.

FIG. 11 illustrates another example of a graphical user interface that can be used by a user to prescribe a neural dose for delivery by the pulse generator of FIG. 2.

FIG. 12 schematically illustrates an example of a method of adjusting energy delivered to neural tissue by the pulse generator of FIG. 2.

### DETAILED DESCRIPTION

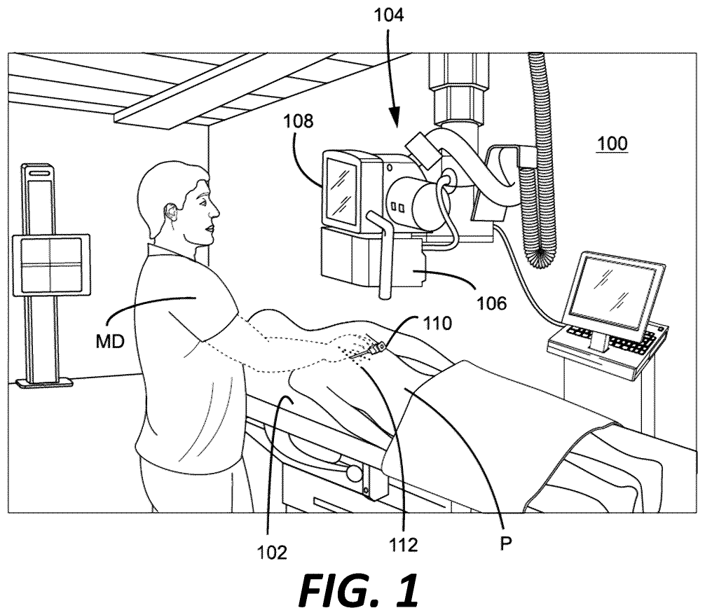

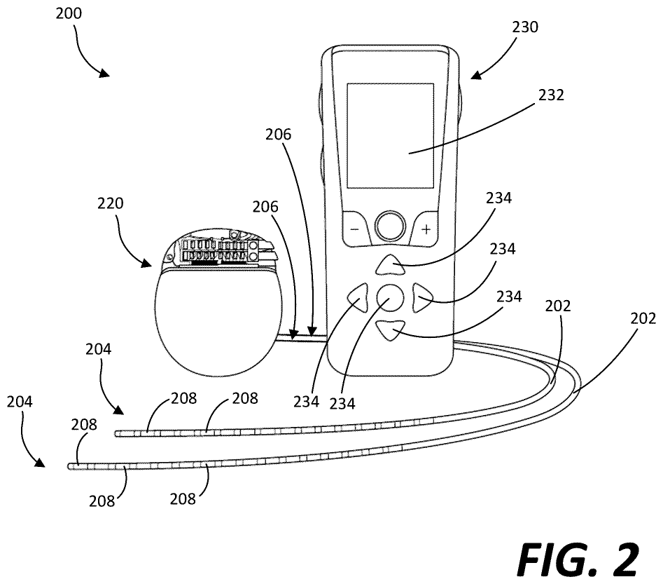

FIG. 1 illustrates an example of a system 100 for performing a medical procedure to implant a pulse generator having one or more leads. The pulse generator and the one or more leads can be included as part of a kit 200, which is shown in more detail in FIG. 2.

The system 100 includes a table 102 on which a patient P rests face down. The patient P's back is exposed for a physician MD to insert an epidural needle 110 for positioning the one or more leads proximate to and/or inside of a targeted area of an epidural space of the patient P's spine. In this example, the epidural needle 110 is used for positioning percutaneous leads, which can be temporary leads used for a trial procedure, or can be permanent percutaneous leads.

The epidural needle 110 is inserted into the skin, and through the paravertebral muscles until it reaches the lamina next to the spinous process located just below a selected target location in the epidural space of the patient P's spine. The epidural needle 110 is then advanced through the ligamentum flavum and into the epidural space. The epidural space is located between the dura mater and the wall of the spinal canal. The epidural space contains fat, veins, arteries, spinal nerve roots and lymphatics. A distal end of at least one lead having electrical contacts is implanted in the epidural space for releasing electrical signals to stimulate the nerves and tissues near the epidural space to interfere with and/or block pain signals that are received by the brain to mitigate pain and other symptoms felt by the patient P. As will be described in more detail, the electrical signals are released based on a neural dose prescription.

The system 100 includes a fluoroscopy system 104 that has at least an imaging device 106 that captures fluoroscopy images (i.e., X-ray images) of the patient P's spine, and a display device 108 that displays the fluoroscopy images for viewing by the physician MD. The physician MD can view the fluoroscopy images for guidance when inserting the epidural needle 110 into the patient P's epidural space, and while positioning the leads into the patient P's epidural space.

As an illustrative example, the physician MD inserts the epidural needle 110 into a targeted area of the epidural space of the patient P's spine, and then threads one or more leads through the epidural needle 110 for positioning the distal ends of the leads inside the epidural space. In other examples, the leads can be implanted proximate the targeted area of the epidural space for stimulating nearby neural tissue while remaining outside of the epidural space.

In the example shown in FIG. 1, a pattern of markings 112 are drawn on the patient P's back. The pattern of markings 112 can be used by the physician MD as a path for guiding the insertion of the epidural needle 110 into the patient P's spine at a correct angle. The pattern of markings 112 can be drawn by using a device described in U.S. patent application Ser. No. 17/113,232, filed Dec. 7, 2020, the entirety of which is hereby incorporated by reference.

FIG. 2 illustrates a kit 200 for spinal cord stimulation that can be implanted to provide a therapeutic effect such as to reduce or block pain symptoms experienced by the patient P. As shown in FIG. 2, the kit 200 includes a pulse generator 220, and leads 202 that connect to the pulse generator. In some examples, the leads 202 physically connect to the pulse generator 220. In alternative examples, the leads 202 wirelessly connect to the pulse generator 220 such as through Bluetooth, Wi-Fi, Zigbee, near-field communication (NFC), or other wireless communications protocols.

The kit 200 also includes a remote control 230 that can be operated by the patient P to wirelessly control the operation of the pulse generator 220 and/or the leads 202 once implanted. Examples of the remote control 230 can include a smartphone, tablet computer, or other portable electronic device that can receive inputs from the patient for controlling operation of the pulse generator 220 and/or the leads 202.

In the example illustrated in FIG. 2, the kit 200 includes a pair of percutaneous leads that connect to the pulse generator 220 via a wired or wireless connection. The pulse generator 220 is programmed to generate electrical signals for transmission through the leads 202 and for release by contacts 208 positioned toward the distal ends of the leads 202.

The contacts 208 are configured to release the electrical signals generated by the pulse generator 220 at a targeted area in the epidural space of the patient P's spine to reduce or block pain signals that are sent to the brain. As will be described in more detail, the electrical signals are generated by the pulse generator 220 based on a neural dose prescription. The neural dose prescription controls and/or limits the release of electrical signals for the stimulation of nerves and ganglia near the epidural space to interfere with and/or block pain signals that are transmitted by the spinal cord of the central nervous system to the brain. The neural dose prescription quantifies a discrete amount of energy delivered and/or transferred to tissues of the nervous system by the pulse generator 220.

In some instances, the neural dose prescription is prescribed by the physician MD who implanted the pulse generator 220. In other instances, the neural dose prescription is prescribed by another medical professional trained in spinal cord stimulation, or by a pharmacist. Additional persons who can prescribe the neural dose prescription are contemplated.

In some examples, the leads 202 are temporary percutaneous leads that are implanted during a temporary trial for spinal cord stimulation. In other examples, the leads 202 are permanent percutaneous leads that are implanted after completion of a successful temporary trial for spinal cord stimulation. In alternative examples, the leads 202 can include permanent paddle leads, as well as other types of leads that are suitable for spinal cord stimulation.

Each of the leads 202 has a body that extends between a distal end 204 and a proximal end 206. In some examples, the body has a tubular shape of a uniform diameter and circumference that allows the leads 202 to easily slide through the epidural needle 110. In some examples, the distal ends 204 of the leads 202 are configured for placement at the targeted area in the epidural space of the patient P's spine. In other examples, the leads 202 are configured for placement proximate to the targeted area of the epidural space, while remaining outside of the epidural space. The proximal ends 206 of the leads 202 connect to the pulse generator 220. In some examples, the proximal ends 206 of the leads 202 wirelessly connect to the pulse generator 220 such as through a passive or active antenna that can be attached to the leads 202 for communicating with the pulse generator.

The distal ends 204 of the leads 202 include contacts 208 that are each configured to receive the electrical signals from the pulse generator 220, and to release the electrical signals such that the electrical signals emanate from inside the epidural space to stimulate nearby neural tissues at the targeted area of the epidural space to provide a therapeutic treatment such as to reduce or block pain signals, and/or to mitigate symptoms caused by asthma, angina, sexual dysfunction, urological and fecal continence disorders, and other types of diseases and disorders. Alternatively, the contacts 208 can release the electrical signals from outside the epidural space to stimulate neural tissue near the targeted area of the epidural space.

In some examples, the contacts 208 are evenly spaced apart. The number of contacts 208 included on each of the leads 202 may vary. The leads 202 can include at least one contact, or can include a plurality of contacts ranging anywhere from 2-20 contacts, or more.

In examples where the kit 200 is used for a trial spinal cord stimulation procedure, the pulse generator 220 is an external device that is carried or worn by the patient P. In such examples, the proximal end 206 of each of the leads 202 is configured to extend outside of the patient P's body for connection to a port included on the pulse generator 220. Alternatively, the proximal ends 206 of the leads 202 can wirelessly connect to the pulse generator 220 such as through a passive or active antenna that can be attached to the leads 202 for communicating with the pulse generator which can remain outside of the body.

Alternatively, in examples where the kit 200 is used for a regular or permanent spinal cord stimulation procedure, the pulse generator 220 is implanted under a skin surface of the patient P. In such examples, the proximal end 206 of each of the leads 202 connects to a port included on the pulse generator 220 and remains inside the patient P's body. Alternatively, the leads 202 can wirelessly connect to the pulse generator 220. In some examples, the pulse generator 220 remains outside of the body while the leads 202 are implanted inside the body proximate to and/or inside of a targeted area of an epidural space.

The kit 200 can further include the remote control 230 that wirelessly communicates with the pulse generator 220 to control operation of the pulse generator 220. As shown in FIG. 2, the remote control 230 includes a display screen 232 and input devices 234. In the example illustrated in FIG. 2, the input devices 234 are physical buttons that are depressible. In alternative examples, the display screen 232 is a touchscreen such that the display screen 232 also acts as an input device. As will be described in more detail, the display screen 232 and the input devices 234 can be used by the patient P to select the neural dose prescription.

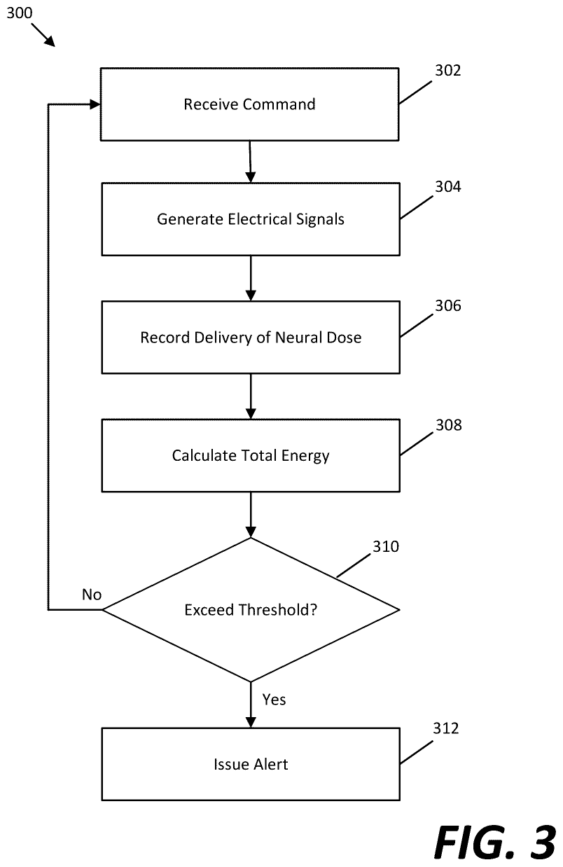

FIG. 3 illustrates an example of a method 300 of monitoring spinal cord stimulation during therapeutic treatment. The method 300 can be performed by the pulse generator 220 of the kit 200 shown in FIG. 2. The method 300 can be performed to calculate and monitor energy released by the leads of the pulse generator 220 emanating from inside the epidural space, or outside of the epidural space depending on where the leads are implanted, over a predetermined period of time. This information can be later used to prescribe a neural dose that does not exceed a threshold amount of energy determined as safe with respect to various neural tissues.

As shown in FIG. 3, the method 300 includes an operation 302 of receiving a command that identifies a neural dose prescription. In some examples, the command is received by the pulse generator 220 from the remote control 230 when operated by the patient P.

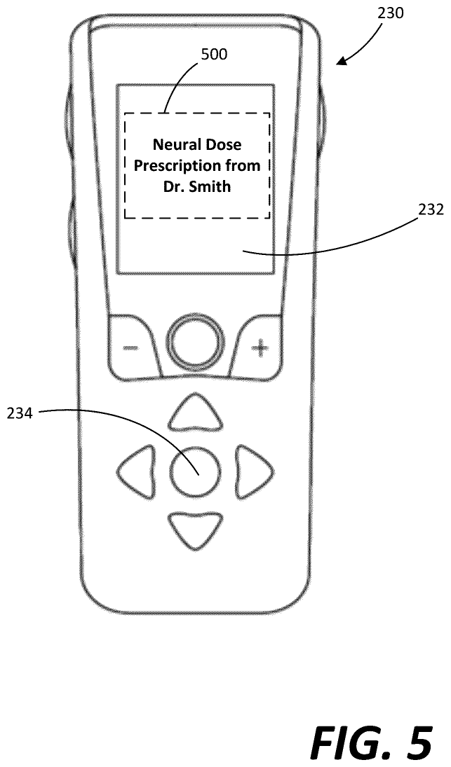

FIG. 5 illustrates an example in which the display screen 232 of the remote control 230 displays a neural dose prescription 500. The patient P can select the neural dose prescription 500 by using an input device 234. When the neural dose prescription 500 is selected, the remote control 230 generates the command that is received by the pulse generator 220.

The neural dose prescription 500 limits the amount of energy to be delivered proximate to and/or inside of a targeted area of the epidural space of the patient P's spine by the pulse generator 220 via the contacts 208 on the distal ends 204 of the leads 202. For example, the neural dose prescription 500 sets a discrete amount of energy (i.e., an effective neural dose) for delivery to the neural tissue near the targeted area of the epidural space to effectively diminish or eliminate pain and other undesirable symptoms felt by the patient P. The neural dose prescription 500 is prescribed to have a lowest effective amount of energy for interfering with or blocking pain signals received by the brain from the central nervous system. The neural dose prescription 500 that provides the lowest effective amount of energy is customizable based on the type of symptom (e.g., pain, wheezing, incontinence, etc.) felt by the patient P, the severity of the symptom, and/or the health condition of the patient P.

As an illustrative example, a neural dose can be prescribed for multiple types of neurostimulation including dorsal root ganglion (DRG) stimulation for pain relief, spinal cord stimulation (SCS) for treating asthma symptoms (e.g., wheezing, shortness of breath, chest tightness, and coughing), SCS for mitigating pain and discomfort caused by angina, SCS for treating pain caused by diabetic neuropathy, SCS for treating sexual dysfunction, peripheral nerve stimulation for treating pain, sacral nerve stimulation for treating urological or fecal continence disorders, peripheral nerve stimulation for treating sexual dysfunction, vagus nerve stimulation for treating epilepsy, depression, and other mental disorders, and deep brain stimulation. Additional types of neurostimulation are contemplated.

As an illustrative example, a neural dose for quantifying a discrete amount of energy released by the electrical signals generated from the pulse generator 220 can be calculated in accordance with Equation 1.

neural dose=A²×I×T (1)

where A is amplitude, I is impedance, and T is time. The amplitude A can be set by the software installed on the pulse generator 220. The impedance I can be measured at current driver circuitry of the pulse generator. The time T is the actual duration of the delivered pulses, measured at the pulse generator 220.

As another illustrative example, the time T can include a cycling ratio of the pulse generator 220, such that time T is calculated in accordance with Equation 2.

$$T = Pulse Width \times Rate \times ( \frac{Burst ON}{Burst ON + Burst OFF} ) \times ( \frac{Dose ON}{Dose ON + Dose OFF} ) \qquad ( 2 )$$

Alternatively, the time T is calculated in accordance with Equation 3.

$$T = Pulse Width \times Rate \times ( \frac{Burst Width}{Burst IPP} ) \times ( \frac{Dose ON}{Dose ON + Dose OFF} ) \qquad ( 3 )$$

In this example, the cycling ratio includes expanded dimensions that can affect the neurophysiological response by the patient. For example, the pulse generator 220 can provide more than just a simple ratio of on/off cycles. For instance, the pulse generator 220 can generate waveforms using both micro and macro cycling parameters, which are beneficial to the neural therapy. The pulse generator 220 can manage these cycling dimensions to manage the waveforms and deliver the prescribed neural dose.

As an illustrative example, typical nerve stimulation pulses are in the microsecond (μs) time scale (e.g., 30 to 1,000 μs). Introducing a set of cycling parameters that control the micro-cycle can create a trail of pulses based on the duration of ON and OFF time in the millisecond scale (ms). For instance, if a user were to want a sequence of ten 1,000 μs pulses at 500 Hz and then turn off, and then repeat the sequence 40 times per second, the pulse generator 220 can be programmed to a micro-cycle of 10 ms ON, 15 ms Off. This results in an inter-burst period of 25 ms, which is 40 Hz. The range of micro-cycles can be from about 5 ms to 1,000 ms. Neurophysiological equipment has long shown the benefits of burst pulses.

Additionally, the pulse generator 220 can also have an additional dimension of cycling that generates a macro-dose parameter. The macro-dose value range can be from about 1 second to about 10,000 seconds. As an example, the macro-dose parameter can be defined in seconds such as 60 seconds on, and 180 seconds off. In further examples, the macro-dose parameter can be defined in minutes and/or hours such as 30 minutes on, and 180 minutes off, or 0.5 hours on, and 3 hours off. In further embodiments, there might be additional dimensions to cycling that include time scales for hours, days, weeks, months, or years.

The benefits of a macro-dose may show up in relief from neuroplasticity and/or improved battery life. For example, the pulse generator 220 and/or the leads 202 can include an internal power source such as a rechargeable or disposable battery for generating the electrical signals released by the contacts 208. The life expectancy of the internal power source can be influenced by the micro and macro cycling ratios such that by increasing these cycling ratios, which causes switching between on and off states to occur more frequently, can increase battery load stress, and thereby decrease the life expectancy of the internal power source.

In view of the forgoing, Equation 4 illustrates an example of calculating a neural dose over a time period of 6 minutes, where the neural dose has a micro-cycle of 10 ms ON, 15 ms Off, and a macro-cycle of 30 seconds on, and 330 seconds off.

$$Neural Dose = 2 {mA}^{2} \times 500 Ω \times 300 µs \times 60 Hz ( \frac{10 ms}{10 ms + 15 ms} ) \times ( \frac{30 s}{30 s + 330 s} ) = 1.2 µWatt / 6 min s = 0.2 µW / min = 3.3 nW / sec$$

In another example, the neural dose prescription can include a calculation of the energy released multiplied by a scaling factor for simplifying its use and to return a unit-less value, as shown in Equation 5 provided below. The scaling factor may be linear, logarithmic, or exponential. As an illustrative example, a low neural dose prescription has a unit-less value of 1 and a high neural dose prescription has a unit-less value of 25.

Neural Dose=Energy×Y(where Y is an arbitrary scaling factor) (5)

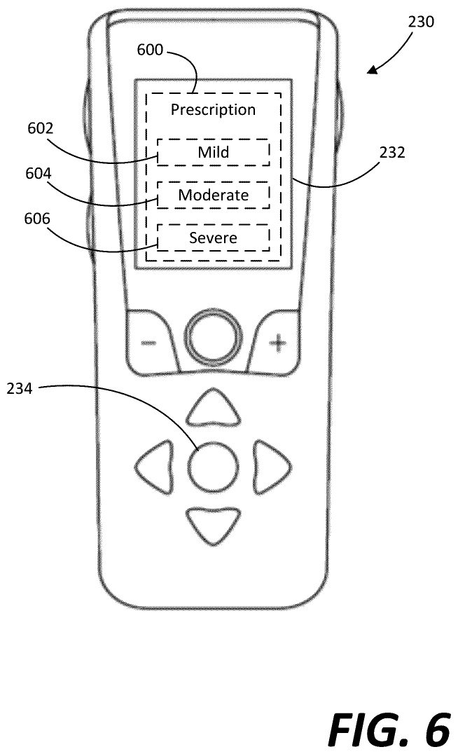

FIG. 6 illustrates an example of the display screen 232 of the remote control 230 displaying a neural dose prescription 600 that includes different dosage levels that each vary a discrete amount of energy delivered to the epidural space of the patient P's spine by the pulse generator 220 via the contacts 208 on the distal ends 204 of the leads 202. In the example illustrated in FIG. 6, the different levels of dosage include a mild dosage level 602, a moderate dosage level 604, and a severe dosage level 606, that are each configured to reduce or block pain symptoms based on the level or severity of pain and symptoms experienced by the patient P.

Each of the dosage levels 602-606 provides a different discrete amount of energy for delivery to the epidural space of the patient P's spine. In the example shown in FIG. 6, the mild dosage level 602 provides the least amount of energy, whereas the severe dosage level 606 provides the largest amount of energy. The dosage levels of the neural dose prescription 600 are customizable based on the pain and symptoms experienced by the patient P, as well as the health condition of the patient P. While FIG. 6 illustrates the neural dose prescription 600 as including three different dosage levels, it is contemplated that in alternative examples there may be more than three different dosage levels, or there may be fewer than three dosage levels.

Returning back to FIG. 3, the method 300 next includes an operation 304 of generating electrical signals based on the neural dose prescription (or dosage level) indicated in the command received in operation 302. The electrical signals are generated by the pulse generator 220 for release by the contacts 208 positioned on the distal ends 204 of the leads 202. The electrical signals are generated according to the neural dose prescription to interrupt pain signals received by the brain from the nerves near the epidural space of the spine.

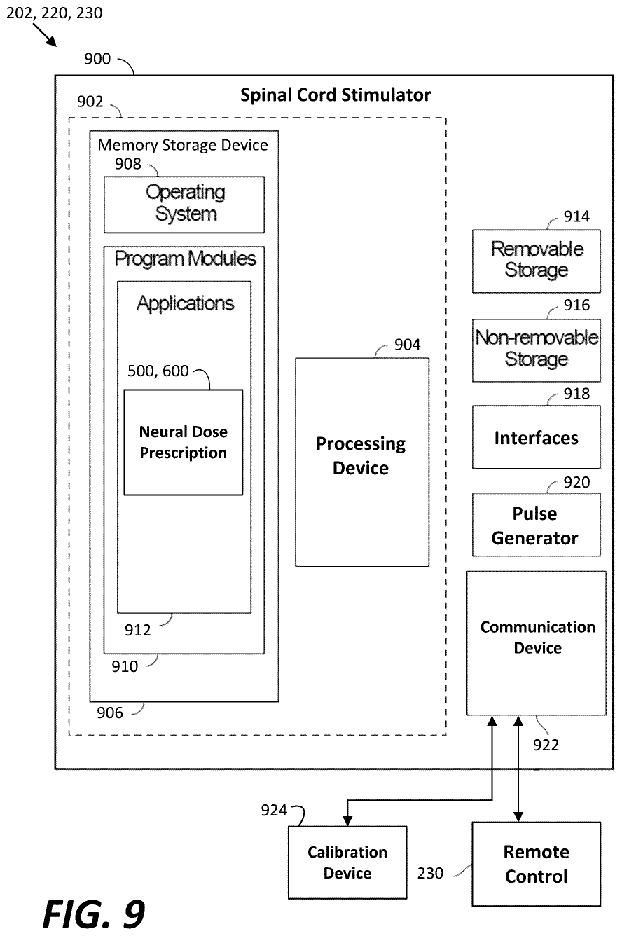

Next, the method 300 includes an operation 306 of recording delivery of the neural dose prescription. The delivery of the neural dose prescription can be recorded or stored on a data storage device (see FIG. 9) of the pulse generator 220. In some examples, the delivery of the neural dose prescription is recorded as a numerical count that can identify an amount of energy delivered to the epidural space of the patient P's spine based on the amount of energy quantified by the neural dose prescription 500 (see FIG. 5), or based on the amount of energy quantified by the dosage levels 602-606 of the neural dose prescription 600 (see FIG. 6).

Next, the method 300 includes an operation 308 of calculating a total amount of energy delivered to the epidural space of the patient P's spine over a predetermined period of time based on recorded deliveries of the neural dose prescriptions (or dosage levels). Operation 308 can include summing the amount of energy for each instance the neural dose prescription 500 is delivered to the epidural space, or summing the amount of energy for each instance a dosage level 602-606 of the neural dose prescription 600 is delivered to the epidural space. The predetermined period of time can be set for 24 hours, 7 days, 30 days, 60 days, 90 days, or a different period of time. Thus, the total amount of energy calculated in operation 308 can be calculated for a single day, a single week, 30 days, 60 days, 90 days, or other period of time.

Next, the method 300 includes an operation 310 of determining whether the total amount of energy (i.e., calculated in operation 308) is outside of upper and/or lower limits of a range of energy set for the predetermined period of time. The upper and/or lower limits of the range can be set by the physician MD or another trained medical professional such as a pharmacist. The upper and lower limits of the range can be used to determine whether the neural dose prescription is effective, and can be used to determine whether the neural dose prescription should be adjusted. In some examples, the range includes both upper and lower limits. In alternative examples, the range includes only an upper limit such that it is a threshold.

When the total amount of energy is within the upper and lower limits of the range of energy (i.e., “No” at operation 310), the method 300 can return to operation 302. In such examples, the method 300 can continue to repeat the operations 302-310 until the total amount of energy is outside of the upper and lower limits of the range (i.e., “Yes” at operation 310).

When the total amount of energy is outside of the upper and lower limits of the range (i.e., “Yes” at operation 310), the method 300 can proceed to an operation 312 of issuing an alert. In some examples, the alert is displayed on the display screen 232 of the remote control 230. In some examples, the alert can include a sensory indicator such as a vibration, beeping noise, or flashing light generated on the remote control 230 to notify or issue a warning to the patient P that the neural dose prescription is not effective. In some further examples, the alert includes a request for the patient P to schedule follow up visit with the physician MD who implanted the pulse generator 220, or with another trained medical professional such as a pharmacist.

In other examples, the alert is sent directly to the physician MD who implanted the pulse generator 220, or to another trained medical professional such as a pharmacist. In such examples, the alert can be sent as a text message, an email, or other type of notification that can be sent directly to a computing device operated by the physician MD or other trained medical professional such as through telecommunications networks and/or the internet.

As an illustrative example, when the total amount of energy exceeds an upper limit of the range (i.e., the threshold), the physician MD can recommend a follow up medical procedure to adjust the position of the leads 202 in the epidural space of the patient P's spine. In this example, the distal ends 204 of the leads 202 where the contacts 208 are positioned may have moved or shifted their position such that the contacts 208 are no longer effectively releasing the electrical signals (based on the neural dose prescription) to an optimal location inside or near the epidural space. This can cause the patient P to select the neural dose prescription 500, or the dosage levels 602-606 of the neural dose prescription 600 (especially the severe dosage level 606) too frequently, which may cause an unhealthy amount of electrical energy to be released into the epidural space of the patient P's spine. The follow up medical procedure can be performed to reposition the contacts 208 to more effectively release the electrical signals at the optimal location in the epidural space of the patient P's spine to more effectively interfere with and/or block the pain signals in accordance with the neural dose prescription.

As another illustrative example, when the total amount of energy exceeds an upper limit of the range (i.e., the threshold), the physician MD or another trained medical professional such as a pharmacist can adjust the neural dose prescription 500, or the dosage levels 602-606 of the neural dose prescription 600 to lower the amount of energy delivered each time the neural dose prescription (or dosage level) is administered by the pulse generator 220. In this example, the pulse generator 220 can receive an update from a calibration device 924 (see FIG. 9), that is separate from the pulse generator 220. The calibration device 924 can upload and/or program the modification of the neural dose prescriptions 500, 600 onto at least one memory storage device 906 of the pulse generator 220 (see FIG. 9) through a wireless connection.

The update modifies the neural dose prescription 500, or the dosage levels 602-606 of the neural dose prescription 600 to adjust the lowest effective amount of energy set for the neural dose prescription (or dosage level). In this manner, the amount of energy delivered to the epidural space from the electrical signals released by the contacts 208 can be reduced to avoid potential harm to the patient P over extended use of the pulse generator 220.

As another illustrative example, when the total amount of energy is below a lower limit of the range, the physician MD can recommend removal of the pulse generator 220 because it may no longer be needed to reduce or mitigate pain felt by the patient P. In such examples, the intractable pain previously felt by the patient (and the reason for implanting the pulse generator 220) may have been cured by other means such as additional surgeries, natural healing, new medications and therapies, and the like. When the pulse generator 220 is no longer being used by the patient P (as evidenced by the total amount of energy being below the lower limit of the range), the pulse generator 220 can be removed.

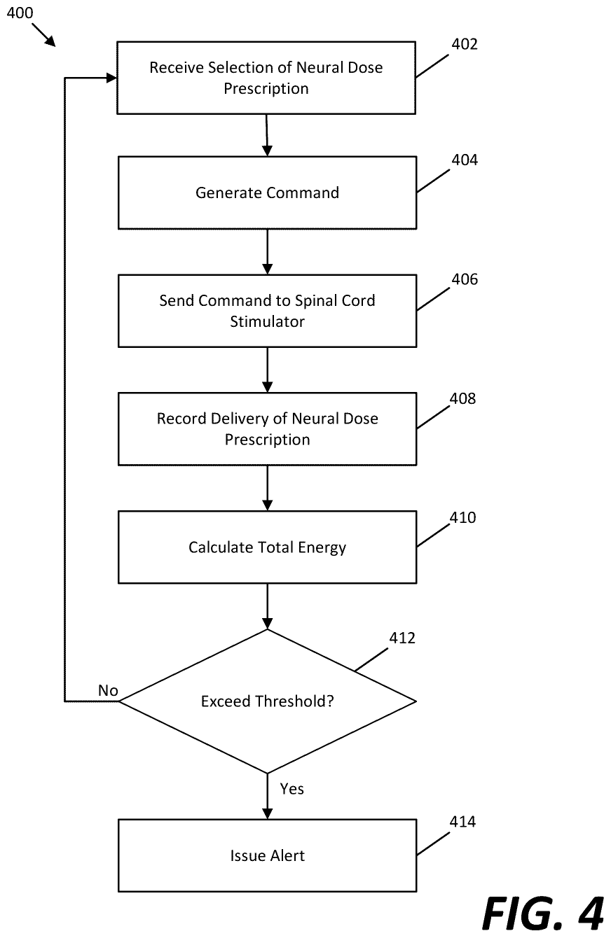

FIG. 4 illustrates another example of a method 400 of monitoring spinal cord stimulation during therapeutic treatment. In some examples, the method 400 is performed by the remote control 230. As shown in FIG. 4, the method 400 includes an operation 402 of receiving a selection of the neural dose prescription, or a dosage level 602-606 of the neural dose prescription 600. As an illustrative example, the patient P can use the input devices 234 to navigate through various screens displayed on the display screen 232, and to select the neural dose prescription 500, or a dosage level 602-606 of the neural dose prescription 600. The neural dose prescription 500 and dosage levels 602-606 of the neural dose prescription 600 are prescribed to provide the lowest effective amount of energy to interfere with or block pain signals.

Next, the method 400 includes an operation 404 of generating a command based on the selection of the neural dose prescription in operation 402. Next, the method 400 includes an operation 406 of sending the command to the pulse generator 220. The command is sent via a wireless connection with the pulse generator 220 (see communication device 922 shown in FIG. 9 and described in more detail below). In some examples, operation 406 includes a step of receiving confirmation that the command is received by the pulse generator 220. The command causes the pulse generator 220 to generate electrical signals for interfering with or blocking pain signals based on the neural dose prescription 500, 600.

Next, the method 400 includes an operation 408 of recording delivery of the neural dose prescription. In some examples, the remote control 230 records delivery of the neural dose prescription based on sending the command to the pulse generator 220 in operation 406. In some examples, the remote control 230 records delivery of the neural dose prescription based on confirmation that the command is received by the pulse generator 220.

Next, the method 400 includes an operation 410 of calculating a total amount of energy delivered to the epidural space of the patient P's spine over a period of time based on recorded deliveries of the neural dose prescriptions (or dosage levels). Operation 410 can be substantially similar to the operation 308 described for the method 300. For example, operation 410 can include summing the amount of energy for each instance the neural dose prescription 500 is delivered to the epidural space, or summing the amount of energy for each instance a dosage level 602-606 of the neural dose prescription 600 is delivered to the epidural space.

Next, the method 400 includes an operation 412 of determining whether the total amount of energy (i.e., calculated in operation 410) is outside of an upper or lower limit of a threshold specified for a predetermined period of time. Operation 412 can be substantially similar to the operation 310 described for the method 300. The upper and/or lower limits of the threshold can be set by the physician MD or another trained medical professional including a pharmacist. The upper and lower limits of the threshold can be used to determine whether the neural dose prescription is effective, and/or whether it should be adjusted.

When the total amount of energy is within an upper and/or lower limits of the threshold (i.e., “No” at operation 412), the method 400 can return to operation 402, and can continue to repeat the operations 402-412 until the total amount of energy exceeds the threshold.

When the total amount of energy is outside of an upper or lower limit of the threshold (i.e., “Yes” at operation 412), the method 400 can proceed to an operation 414 of issuing an alert. In some examples, the alert is displayed on the display screen 232 of the remote control 230. In some examples, the alert can include a sensory indicator such as a vibration, beeping noise, or flashing light generated on the remote control 230 to notify or issue a warning to the patient P. In some further example, the alert includes a request for the patient P to schedule follow up visit with the physician MD who implanted the pulse generator 220, or with another medical professional trained in spinal cord stimulation, or with a pharmacist.

In other examples, the alert is sent directly to the physician MD who implanted the pulse generator 220, or to another trained medical professional such as a pharmacist. In such examples, the alert can be sent as a text message, an email, or other type of notification that can be sent directly to a computing device operated by the physician MD or other trained medical professional such as through telecommunications networks and/or the internet.

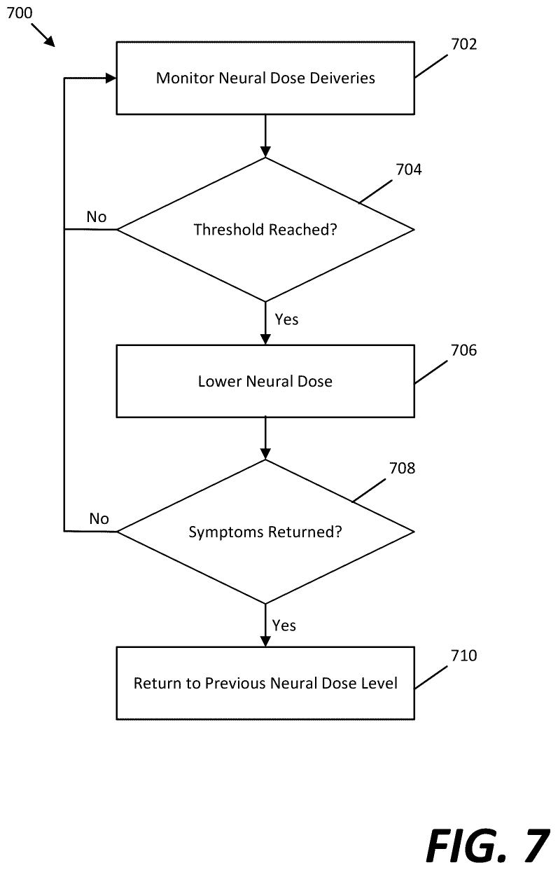

FIG. 7 schematically illustrates an example of a method 700 of performing a titration of a neural dose prescription. In some instances, the method 700 is performed by the pulse generator 220. Alternatively, the method 700 can be performed by the remote control 230. The method 700 includes an operation 702 of monitoring neural dose deliveries. Operation 702 can include recording delivery of neural doses over a period of time. In some examples, deliveries of the neural doses are recorded as numerical counts that identify an amount of energy delivered to the neural tissue surrounding the epidural space of the patient P's spine.

Next, the method 700 includes an operation 704 of determining whether a threshold has been reached. The threshold can include a total amount of energy, or a total number of neural doses delivered to the neural tissue surrounding the epidural space of the patient P's spine based on recorded deliveries of the neural doses. Alternatively, the threshold can include a predetermined period of time such as 1 week, 2 weeks, 1 month, 3 months, etc.

When operation 704 determines that the threshold has not been reached (i.e., “No” in operation 704), the method 700 can return to operation 702 of monitoring the neural dose deliveries. When operation 704 determines that the threshold is reached (i.e., “Yes” in operation 704), the method 700 proceeds to an operation 706 of lowering the neural dose such that less energy is delivered to the neural tissue surrounding the epidural space of the patient P's spine. Operation 706 can include lowering the neural dose by a predetermined amount such as an amount prescribed by the physician MD who implanted the pulse generator 220, or an amount prescribed by another trained medical professional such as a pharmacist.

Next, the method 700 includes an operation 708 of determining whether the symptoms experienced by the patient P have returned. In some examples, operation 708 can include receiving feedback from the patient P via the display screen 232 and/or the input devices 234 of the remote control 230 that indicates the patient is experiencing a return of the symptoms. In some examples, the display screen 232 can display a prompt requesting the patient P to provide the feedback. The prompt can be generated for display on the remote control 230 after a predetermined period of time has expired from when the neural dose is lowered.

In some examples, the pulse generator 220 can include a sensor that detects a return of the symptoms experienced by the patient. In some examples, operation 708 can include using the sensor to directly detect a return of the symptoms such as a microphone or other type of sensor that detects that the patient P is wheezing, having shortness of breath, chest tightness, and/or is coughing (when the pulse generator 220 is being used to stimulate the neural tissue surrounding the epidural space to treat symptoms caused by asthma).

Alternatively, operation 708 can include indirectly detecting a return of the symptoms experienced by the patient P. For example, the pulse generator 220 or the remote control 230 can include a sensor that tracks the patient P's movements such as an accelerometer, a gyroscope, a step counter, and the like. When the patient P is detected as taking fewer steps or as being less active, this can indirectly indicate that the pain symptoms have returned.

When operation 708 determines that the symptoms experienced by the patient P have returned (i.e., “Yes” in operation 708), the method 700 proceeds to an operation 710 of returning to a previous neural dose level. For example, the neural dose can be returned to the previous amount before the neural dose was lowered in operation 706. Otherwise, when operation 708 determines that the symptoms experienced by the patient P have not returned (i.e., “No” in operation 708), the method 700 can repeat operations 702-708 to continually lower the neural dose until the symptoms experienced by the patient P return (i.e., “Yes” in operation 708), at which point the method 700 proceeds to operation 710 of returning to a previous neural dose level. In this manner, the method 700 performs a titration of a neural dose prescription until a lowest effective amount of energy is reached for mitigating the symptoms of the patient P.

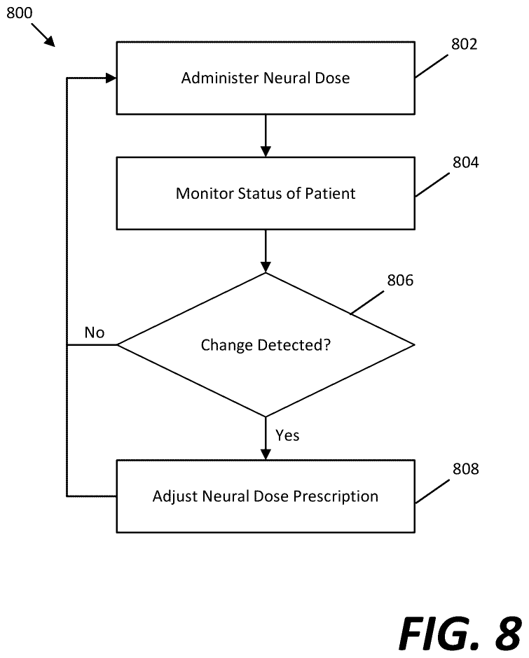

FIG. 8 schematically illustrates an example of a method 800 of automatically adjusting a neural dose based on a status of the patient P. The method 800 can be performed by the remote control 230 such as when the remote control 230 is a smartphone, tablet computer, or other portable electronic device carried by the patient P that receives inputs from the patient P for controlling operation of the pulse generator 220 and/or the leads 202. Alternatively, the method 800 can be performed by the pulse generator 220. The method 800 includes an operation 802 of administering a neural dose, as described in the previous examples.

Next, the method 800 includes an operation 804 of monitoring a status of the patient P. For example, operation 804 can include monitoring a physical activity of the patient P such as whether the patient P is resting, or is engaging in physical activity such as walking, running, riding a bicycle, and the like. As described above, the pulse generator 220 or the remote control 230 can include a sensor that tracks the patient P's movements such as an accelerometer, a gyroscope, a step counter, and the like. In some further examples, geolocating or positioning systems can be enabled on the remote control 230, such as Global Positioning System (GPS) and cell tower triangulation, and these systems can be used to track the movement of the patient P.

Alternatively, operation 804 can include monitoring an environment of the patient P. For example, operation 804 can include monitoring the weather of the location of the patient P including the temperature, humidity, pollen count, and other environmental factors.

Next, the method 800 includes an operation 806 of determining whether the status of the patient P has changed. When operation 806 determines that the status of the patient P has not changed (i.e., “No” in operation 806), the method 800 can return to operation 802 of administering a neural dose prescription. When operation 806 determines that the status of the patient P has changed (i.e., “Yes” in operation 806), the method 800 proceeds to an operation 808 of adjusting the neural dose based on the detected change in the status of the patient P.

As an illustrative example, when operation 806 determines that the physical activity of the patient P has increased (e.g., the patient P is walking, running, riding a bicycle, and the like), operation 808 can include increasing the neural dose. The increased neural dose can preemptively mitigate an increase in strength of the pain and other symptoms felt by the patient P during and after the increased physical activity. Additional examples of adjusting the neural dose prescription based on detected changes in the patient P's physical activity are contemplated.

As another illustrative example, when operation 806 determines that one or more factors of patient P's environment have changed (e.g., weather forecast indicates a likelihood of increased humidity, decreased temperature, and the like), operation 808 can include increasing the neural dose. The increased neural dose can preemptively mitigate an increase in strength of the pain and other symptoms felt by the patient P due to the increased humidity, which can cause sensitive or inflamed tissues to expand. Also, the increased neural dose can preemptively mitigate an increase in strength of the pain and other symptoms felt by the patient P due to the decreased temperature, which can cause increased joint stiffness due to the body's response to conserve heat by limiting blood flow to the extremities and pumping more blood to the lungs and heart. Additional examples adjusting the neural dose prescription based on detected changes in the patient P's environment are contemplated.

The method 800 can repeat operations 802-808. For example, the method 800 can repeat operations 802-808 to determine whether the status of the patient P has changed again. For example, when operation 806 determines that the physical activity of the patient P has decreased (e.g., the patient P is no longer walking, running, riding a bicycle, and the like), operation 808 can include decreasing the neural dose. As another example, when operation 806 determines that the humidity has decreased or the temperature of the patient P's environment has increased, operation 808 can include decreasing the neural dose.

FIG. 9 schematically illustrates an example of computing components 900 of the pulse generator 220, the remote control 230, and/or the leads 202. As shown in FIG. 9, the computing components 900 include a computing device 902 that includes at least one processing device 904 and at least one memory storage device 906.

The at least one processing device 904 can include a central processing unit (CPU), a microcontroller, and similar devices. The at least one memory storage device 906 can include volatile storage (e.g., random access memory), non-volatile storage (e.g., read-only memory), flash memory, or any combinations thereof.

The at least one memory storage device 906 can include an operating system 908 that is suitable for controlling the operations of the pulse generator 220, the remote control 230, and/or the leads 202. The at least one memory storage device 906 further includes program modules 910 suitable for running one or more software applications 912.

A number of program modules 910 and data files can be stored on the at least one memory storage device 906. While executing by the at least one processing device 904, the program modules 910 may perform various tasks and methods, such as the neural dose prescriptions 500, 600 (and dosage levels 602-606) that are described herein.

As shown in FIG. 9, the neural dose prescriptions 500, 600 can be stored as software applications 912 on the at least one memory storage device 906. The neural dose prescriptions 500, 600 can be uploaded and/or programmed onto the at least one memory storage device 906 before or after the pulse generator 220 and/or the leads 202 are implanted. The neural dose prescriptions 500, 600 are uploaded and/or programmed onto the at least one memory storage device 906 by the calibration device 924 through wireless or wired connections.

Embodiments of the pulse generator 220, the remote control 230, and/or the leads 202 may be practiced with other operating systems and/or application programs not limited to any particular application or system. The basic configuration of the computing device 902 is illustrated by those components shown in FIG. 9. The computing device 902 may have additional features or functionality. For example, the computing device 902 may include additional data storage devices (removable and/or non-removable). The additional storage is illustrated by a removable memory storage device 914 and a non-removable memory storage device 916.

Further embodiments of the pulse generator 220, the remote control 230, and/or the leads 202 may be practiced in an electrical circuit comprising discrete electronic elements, packaged or integrated electronic chips containing logic gates, a circuit utilizing a microprocessor, or on a single chip containing electronic elements or microprocessors. For example, such embodiments may be practiced via a system-on-a-chip (SOC) where each or many of the components illustrated in FIG. 9 may be integrated onto a single integrated circuit.

Such an SOC device may include one or more processing units, graphics units, communications units, system virtualization units and various application functionality all of which are integrated onto the chip substrate as a single integrated circuit. When operating via an SOC, the functionality, described herein, may be operated via application-specific logic integrated with other components of the computing device 902 on the single integrated circuit (chip). Embodiments of the pulse generator 220, the remote control 230, and the leads 202 may also be practiced using other technologies capable of performing logical operations. Also, embodiments of the disclosure may be practiced within a general-purpose computer or in any other circuits or systems.

The term computer readable media as used herein may include non-transitory computer storage media. Computer storage media may include volatile and nonvolatile, removable and non-removable media implemented in any method or technology for storage of information, such as computer readable instructions, data structures, or program modules.

The at least one memory storage device 906, the removable memory storage device 914, and the non-removable memory storage device 916 are computer storage media. Computer storage media may include RAM, ROM, electrically erasable read-only memory (EEPROM), flash memory or other memory technology, or any other article of manufacture which can be used to store data, and which can be accessed by the computing device 902. Any such computer storage media may be part of the computing components 900.

The pulse generator 220 may include one or more interfaces 918 such as ports for physically connecting with the proximal ends 206 of the leads 202, or wireless interfaces for wirelessly connecting to the leads 202. The pulse generator 220 may also include a pulse generator 920 for generating the electrical signals for transmission through the leads 202 and for release by contacts 208 positioned toward the distal ends 204 of the leads 202.

The pulse generator 220, the remote control 230, and/or the leads 202 can include a communication device 922 for providing communication connections with other devices. The communication connections can be accomplished through wireless antennas. Examples of communication connections can include, without limitation, Bluetooth, Wi-Fi, Zigbee, radio frequency (RF), cellular networks (including 4G and 5G networks), and/or transceiver circuitry, universal serial bus (USB), parallel and/or serial ports, and the like.

Communication media may be embodied by computer readable instructions, data structures, program modules, or other data in a modulated data signal, such as a carrier wave or other transport mechanism, and includes any information delivery media. The term “modulated data signal” describes a signal having characteristics set or changed in such a manner as to encode information in the signal. Communication media may include wired media such as a wired network or direct-wired connection, and wireless media such as Bluetooth, Wi-Fi, Cellular (including 4G and 5G networks), radio frequency (RF), infrared, and other wireless media.

The block diagrams depicted in FIG. 9 and elsewhere are just examples. There may be many variations to these diagrams described therein without departing from the spirit of the disclosure. For instance, components may be added, deleted, or modified.

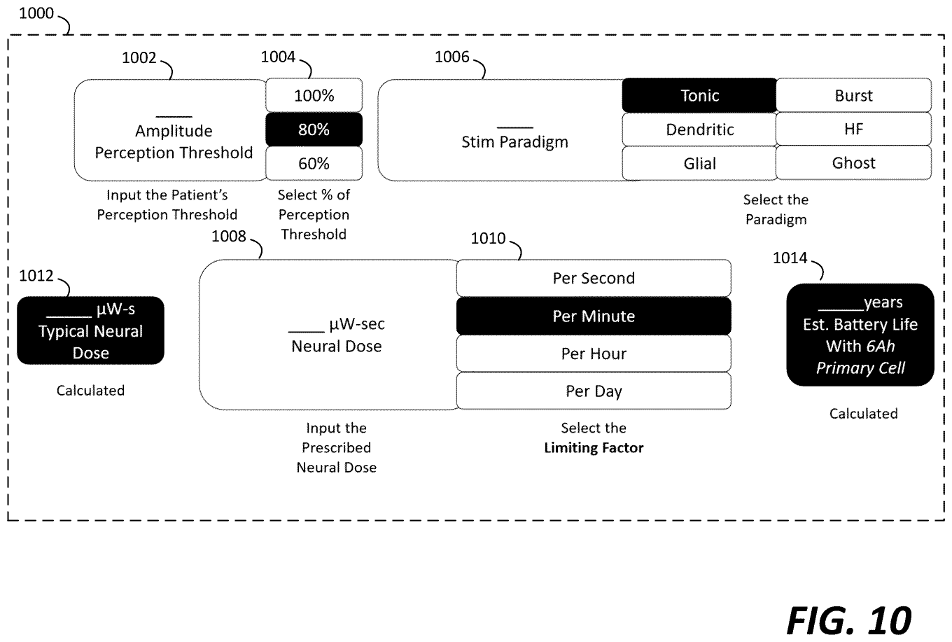

FIG. 10 illustrates an example of a graphical user interface 1000 that can be used by a user such as a physician, a health care provider (HCP), or a technician to prescribe a neural dose. The graphical user interface 1000 displays selectable options 1004 for a target amplitude threshold 1002 set to avoid neural side-effects such as paresthesia, tingling, burning, pain. For example, the selectable options 1004 include percentages of the target amplitude threshold 1002, including but not limited to: 200%, 120%, 100%, 80%, 60%, and the like.

The graphical user interface 1000 can further display one or more types of waveform therapies 1006 for selection by the user. There are multiple studies conducted on many waveform therapies that have shown efficacy for each. These waveform therapies may include, but not limited to: Tonic, Burst, Dendritic Stimulation, High-Frequency, Glial Cell Stimulation, FAST, DTM, Ghost, InstaStim, and the like. The one or more types of waveform therapies 1006 can be program-cycled to prevent neuroplasticity. The graphical user interface 1000 can allow the user to select more than one type of waveform therapy 1006 for the neural dose prescription.

As further shown in FIG. 10, the graphical user interface 1000 includes an input area 1008 for the user to enter a neural dose value. The neural dose value is entered as a form of prescribing total energy (Power×Time). The neural dose value can be entered in units of micro-watt-seconds (μW-s). The graphical user interface 1000 can include an input area 1010 allowing the user to select a scale of the neural dose value over a period of time such as μW-s/second, μW-s/minute, μW-s/hour, μW-s/day, and the like.

As further shown in FIG. 10, the graphical user interface 1000 includes a display area 1012 that can include a total amount of energy delivered to neural tissue based on recorded deliveries of the electrical signals. In some examples, the display area 1012 includes a total amount of energy delivered to the neural tissue based on an elapsed period of time such as a total amount of energy delivered in the previous month, previous week, previous day, and so on. In some further examples, the display area 1012 includes an average amount of energy delivered to the neural tissue such as an average amount per day, per week, per month, and so on. The amount or value displayed inside the display area 1012 can be calculated based on recorded deliveries of the electrical signals in accordance with the examples described above.

The graphical user interface 1000 can also include a display area 1014 that includes an estimated remaining battery life of the pulse generator 220. The remaining battery life can be calculated based on the amount of energy delivered to the neural tissue such that as the amount of energy delivered to the neural tissue increases, the remaining battery life decreases.

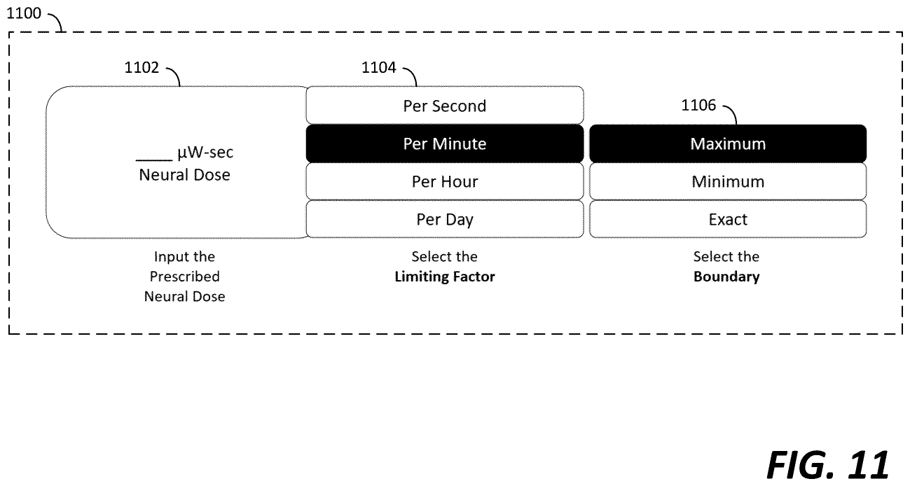

FIG. 11 illustrates another example of a graphical user interface 1100 that can be used by a user such as a physician, a health care provider (HCP), or a technician to prescribe a neural dose. The graphical user interface 1100 includes an input area 1102 for the user to enter a value for the neural dose and an input area 1104 allowing the user to select a scale for the value over a period of time such as μW-s/second, μW-s/minute, μW-s/hour, μW-s/day, and the like. The graphical user interface 1100 further includes an input area 1106 where the user can select a boundary for the value entered in input area 1102 such as whether it is for a maximum neural dose allowed in the period of time selected in the input area 1104, a minimum neural dose allowed in the period of time selected in the input area 1104, or an exact target neural dose.

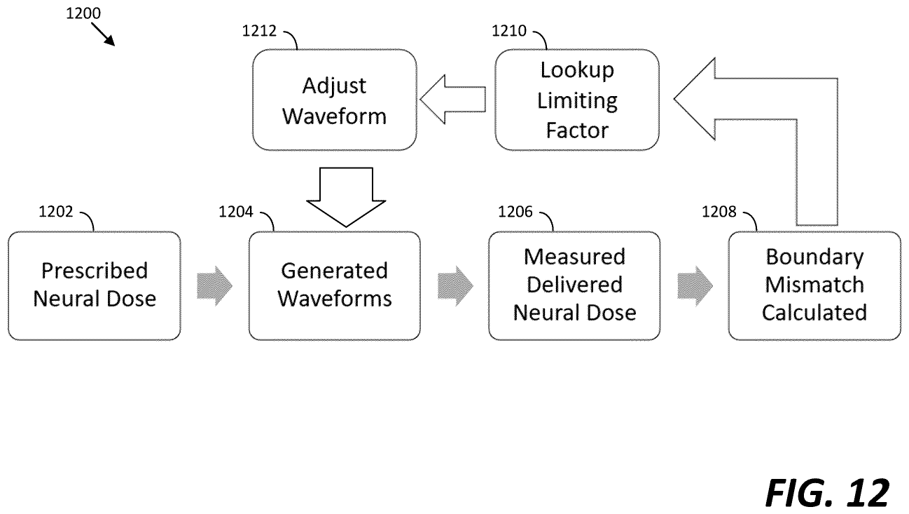

FIG. 12 schematically illustrates an example of a method 1200 of adjusting energy delivered to neural tissue by the pulse generator 220. In some cases, when the neural dose prescription is bounded to be an exact neural dose, the neural dose prescription and the output of energy from the pulse generator 220 are not a perfect match possibly due to impedance, electronic component variation, wear and tear over time, and amplitude settings that the end-user is able to adjust. A neural dose calculator, implemented on either the pulse generator 220 or the remote control 230, can monitor and measure the actual energy delivered to the neural tissue using the techniques and methods described above, compare the actual energy to the neural dose prescription, and then adjust (e.g., increase or decrease) the waveform parameters to ensure compliance with the neural dose prescription.

In some instances, the method 1200 can be performed entirely on the pulse generator 220. As shown in FIG. 12, the method 1200 includes a step 1202 of receiving the neural dose prescription. In accordance with the examples described above, the neural dose prescription can be generated using the graphical user interfaces 1000, 1100 shown in FIGS. 10 and 11, and can be uploaded onto the pulse generator 220 using the calibration device 924.

Next, the method 1200 includes a step 1204 of generating the waveforms to stimulate the neural tissue based on the neural dose description received in step 1202. Thereafter, the method 1200 includes a step 1206 of measuring the delivered neural doses.

Next, the method 1200 includes a step 1208 of calculating a boundary mismatch. Step 1208 can include, for example, comparing the actual energy delivered to the neural tissue (determined in step 1206) to the neural dose prescription (received in step 1202).

When there is a boundary mismatch, the method 1200 can proceed to a step 1210 of looking-up a limiting factor that contributes to the mismatch. For example, step 1210 can include determining at least one of the amplitude, the impedance, the cycling rate, or another parameter as responsible for the boundary mismatch calculated in step 1208.

Next, the method 1200 can proceed to a step 1212 of adjusting the waveforms generated by the pulse generator 220 to reduce and/or eliminate the boundary mismatch. For example, step 1212 can include increasing or decreasing at least one of the amplitude, the impedance, the cycling rate, or another parameter to reduce or eliminate the boundary mismatch. Steps 1204-1212 can be repeated as necessary to reduce or eliminate boundary mismatches that can occur during operation of the pulse generator 220.

As an illustrative example, a user such as physician prescribes a neural dose which permits an amount of energy to be delivered to the neural tissue over a period of time. As an example, the neural dose prescription can include 40 W-s per day, and is set as an exact target neural dose (see input area 1106 of FIG. 11). Table 1 provides illustrative examples of neural doses delivered by different types of waveform therapies 1006.

TABLE 1

| Waveform Therapy | μW-s | μW-s/min | W-s/hour | W-s/day |
|---|---|---|---|---|
| Tonic | 36 | 2,160 | 0.13 | 3.11 |
| Burst | 400 | 24,000 | 1.44 | 34.56 |
| HF | 600 | 36,000 | 2.16 | 51.84 |

As shown in the illustrative example of Table 1, the tonic waveform therapy (3.11) and the burst waveform therapy (34.56) are less than the neural dose prescription of 40 W-s per day. Thus, step 1212 of the method 1200 can include increasing the pulse width and pulse rate of the tonic and burst waveform therapies to match the prescribed neural dose of 40 W-s per day.

As further shown in the illustrative example of Table 1, the high frequency (HF) waveform therapy (51.84) exceeds the neural dose prescription of 40 W-s per day. Accordingly, step 1212 can include adding cycling to decrease the amount of energy delivered by the HF waveform therapy such that it matches the prescribed neural dose of 40 W-s per day.

The various embodiments described above are provided by way of illustration only and should not be construed to be limiting in any way. Various modifications can be made to the embodiments described above without departing from the true spirit and scope of the disclosure.

What is claimed is:

1. A pulse generator, comprising:
at least one processing device; and
at least one memory storage device storing:
a neural dose prescription for releasing electrical signals to stimulate neural tissue; and
instructions which, when executed by the at least one processing device, cause the at least one processing device to:
generate, based on the neural dose prescription, the electrical signals for delivery through a lead, the electrical signals being programmed to mitigate symptoms by stimulating the neural tissue;
record delivery of the electrical signals; and
calculate a total amount of energy delivered to the neural tissue based on recorded deliveries of the electrical signals.

2. The pulse generator of claim 1, wherein the neural dose prescription defines a discrete amount of energy for the electrical signals delivered to the neural tissue.

3. The pulse generator of claim 1, wherein the neural dose prescription includes different dosage levels that each vary an amount of energy delivered to the neural tissue.

4. The pulse generator of claim 3, wherein the different levels of dosage include a mild dosage level, a moderate dosage level, and a severe dosage level.

5. The pulse generator of claim 1, wherein the neural dose prescription provides a lowest effective amount of energy for the electrical signals to mitigate the symptoms.

6. The pulse generator of claim 5, wherein the instructions, when executed by the at least one processing device, further cause the at least one processing device to:
store a threshold defining a maximum amount of energy for delivery to the neural tissue over the predetermined period of time.

7. The pulse generator of claim 6, wherein the instructions, when executed by the at least one processing device, further cause the at least one processing device to:
issue an alert when the total amount of energy delivered to the neural tissue over the predetermined period of time exceeds the threshold.

8. The pulse generator of claim 7, wherein the instructions, when executed by the at least one processing device, further cause the at least one processing device to:
receive an update modifying the neural dose prescription to adjust the lowest effective amount of energy released by the electrical signals.

9. A remote control for operating a pulse generator, the remote control comprising:
at least one processing device; and
at least one memory storage device storing instructions which, when executed by the at least one processing device, cause the at least one processing device to:
generate a command based on a neural dose prescription;
send the command to a pulse generator, the command causing the pulse generator to generate electrical signals, the electrical signals being programmed to mitigate symptoms by stimulating neural tissue;
record delivery of the electrical signals; and
calculate a total amount of energy delivered to the neural tissue based on recorded deliveries of the electrical signals.

10. The remote control of claim 9, further comprising:
a display screen for displaying parameters including the neural dose prescription; and
one or more input devices for navigating through the parameters displayed on the display screen and for selecting the neural dose prescription.

11. The remote control of claim 10, wherein the neural dose prescription includes different dosage levels that each vary an amount of energy delivered to the neural tissue, and the display is configured to display each dosage level for selection by the input device.

12. The remote control of claim 11, wherein the different dosage levels include a mild dosage level, a moderate dosage level, and a severe dosage level.

13. The remote control of claim 9, wherein the instructions, when executed by the at least one processing device, further cause the at least one processing device to:
store a threshold defining a maximum amount of energy for delivery to the neural tissue over the predetermined period of time.

14. The remote control of claim 13, wherein the instructions, when executed by the at least one processing device, further cause the at least one processing device to:
issue an alert when the total amount of energy delivered to the neural tissue over the predetermined period of time exceeds the threshold.

15. A method of performing spinal cord stimulation, the method comprising:
generating electrical signals based on a neural dose prescription, the electrical signals delivering a lowest effective amount of energy for stimulating neural tissue;
recording delivery of the electrical signals; and
calculating a total amount of energy delivered to the neural tissue based on recorded deliveries of the electrical signals.

16. The method of claim 15, further comprising:
determining whether the total amount of energy exceeds a threshold specified for the predetermined period of time.

17. The method of claim 15, further comprising:
receiving an update to adjust the lowest effective amount of energy set by the neural dose prescription.

18. The method of claim 15, wherein the electrical signals are classified based on at least one of a mild dosage level, a moderate dosage level, and a severe dosage level defined by the neural dose prescription, each dosage level varying an amount of energy delivered to the neural tissue.

19. The method of claim 15, further comprising:
issuing an alert when the total amount of energy delivered to the neural tissue over the predetermined period of time exceeds a threshold.

20. The method of claim 15, further comprising:
modifying the neural dose prescription to adjust the lowest effective amount of energy released by the electrical signals.
